(19) United States
(12) Patent Application Publication
(10) Pub. No.: US 2009/0247979 A1
(43) Pub. Date: Oct. 1, 2009

(54) ABSORBENT ARTICLE WITH GRAPHIC ELEMENTS

(75) Inventors: Paula Mary Sosalla, Appleton, WI (US); Brenda Lee Jamroz, Neenah, WI (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: Kimberly-Clark Worldwide, Inc., Neenah, WI (US)

(21) Appl. No.: 12/059,808

(22) Filed: Mar. 31, 2008

(51) Int. Cl. A61F 13/496

(52) U.S. Cl. 604/385.01

(57) ABSTRACT

In an absorbent article for personal wear, an inner surface of the absorbent article is arranged for facing a wearer of the absorbent article and an outer surface is arranged in generally opposed relationship with the inner surface for facing away from the wearer. An absorbent structure is disposed between the inner surface and the outer surface of the absorbent article. An outer graphic element is visible from exterior of the absorbent article and an inner graphic element is visible from interior of the absorbent article. The inner graphic element is separate from the outer graphic element and is substantially identical thereto. The inner and outer graphic elements are in superposed relationship with each other on the absorbent article.

### FIELD

The present invention relates generally to absorbent articles intended for personal wear, and more particularly to such an absorbent article having graphic elements disposed thereon.

### BACKGROUND

Disposable absorbent articles find widespread use as personal care products such as diapers, children's toilet training pants and other infant and child care products, adult incontinence garments and other adult care products, sanitary napkins and other feminine care products and the like, as well as surgical bandages and sponges. These articles absorb and contain body waste and are intended to be discarded after a limited period of use; i.e., the articles are not intended to be laundered or otherwise restored for reuse. Conventional disposable absorbent articles comprise an absorbent body disposed between an inner layer adapted for facing and typically contacting the wearer's skin and an outer layer for inhibiting liquid waste absorbed by the absorbent body from leaking out of the article. The inner layer of the absorbent article is typically liquid permeable to permit body waste to pass therethrough for absorption by the absorbent body.

Disposable absorbent training pants, in particular, are useful in toilet training children. Typically, these training pants are similar to washable, cloth underwear in the manner in which they are put on and worn, yet provide an absorbent function similar to diapers to maintain skin health. Training pants provide a child undergoing toilet training with an absorbent garment that eases the transition from diapers to washable, cloth underwear as they become more confident in their ability to use the toilet independently. Indeed, one important aspect of the total toilet training process is the change from diapers to training pants to help the child understand that he or she should now use the toilet just like adults. Although the use of training pants and positive encouragement from the caregiver has been helpful in the toilet training process, there is still much room for improvement.

One motivational mechanism used to encourage such a transition is the use of disposable training pants that more closely resemble an actual pair of underwear. Specifically, a child is encouraged to wear a garment that resembles underwear worn by older children or resembles other under or outer garments worn by adults. Otherwise, if the child views the training pants as being more similar to a diaper, they are more likely to continue wetting the pants in the same manner as the diaper. To mimic the appearance of underwear and raise the interest of a child during toilet training, training pants are known to have a graphic that is visible from outside the pants, such as by being applied to their exterior or outer surface. The graphic may be in the form of a character, object and/or alphanumeric (e.g., numbers, words, phrases, instructions, etc.). Graphics applied to such disposable articles may also provide visual assistance to the wearer or to a caregiver securing the article on the wearer. Unfortunately, these training pants still lack a more realistic appearance of underwear, particularly because the interior or inner surface of such pants does not resemble an actual pair of underwear.

### SUMMARY

In one embodiment, an absorbent article for personal wear generally comprises an inner surface for facing a wearer of said absorbent article and an outer surface in generally opposed relationship with the inner surface for facing away from the wearer. An absorbent structure is disposed between the inner surface and the outer surface of the absorbent article. An outer graphic element is visible from exterior of the absorbent article and an inner graphic element is visible from interior of the absorbent article. The inner graphic element is separate from the outer graphic element and substantially identical thereto. The inner and outer graphic elements are in superposed relationship with each other on the absorbent article.

In another embodiment, an absorbent article for personal wear generally comprises an inner layer having an inner surface for facing a wearer of the absorbent article and an outer layer in opposed relationship with the inner layer and having an outer surface for facing away from the wearer. An absorbent structure is disposed between the inner layer and the outer layer. An outer graphic element is visible from exterior of the absorbent article and an inner graphic element separate from the outer graphic element is visible from interior of the article. The inner graphic element is associated with the outer graphic element and the inner and outer graphic elements are generally at the same longitudinal and lateral position on the absorbent article.

In yet another embodiment, an absorbent article for personal wear generally comprises a liner having an inner surface for facing a wearer of the absorbent article and an outer cover in opposed relationship with the liner and having an outer surface for facing away from the wearer. An absorbent structure is disposed between the inner layer and the outer layer. An outer graphic element is disposed on the outer cover. An inner graphic element, separate from and substantially identical to the outer graphic element, is disposed on the liner and is in generally superposed relationship with the outer graphic element.

In one embodiment, a method of making an absorbent article having an inner surface for facing a wearer of the absorbent article and an outer surface for facing away from the wearer generally comprises applying a first graphic element to a first substrate of the absorbent article and applying a second graphic element to a second substrate of the absorbent article, the second graphic element being substantially identical to the first graphic element. The absorbent article is assembled with the first and second substrates in generally opposed relationship with each other and an absorbent structure disposed between the first and second substrates. The assembling of the first and second substrates is such that the first graphic element is in generally superposed relationship with the second graphic element.

Other features of the invention will be in part apparent and in part pointed out hereinafter.

### BRIEF DESCRIPTION OF THE DRAWINGS

FIG. 1 is a side perspective of an article of the present invention shown in the form of a pair of training pants having a mechanical fastening system illustrated in a fastened condition on one side of the training pants and in an unfastened condition on the opposite side thereof;

FIG. 2 is a bottom plan view of the training pants of FIG. 1 with the pants in an unfastened, unfolded and laid flat condition, and showing an outer surface of the training pants, which is the surface of the training pants that faces away from the wearer;

FIG. 3 is a top plan view similar to FIG. 2 showing an inner surface of the training pants, which is the surface of the training pants that faces the wearer when worn and with portions cut away to show underlying features;

FIG. 4 is a fragmented bottom plan view of a second embodiment of training pants with the pants in an unfastened, unfolded and laid flat condition, and showing the outer surface of the training pants; and

FIG. 5 is a fragmented top plan view of the training pants of FIG. 4 with the pants in an unfastened, unfolded and laid flat condition, and showing the inner surface of the training pants.

Corresponding reference characters indicate corresponding parts throughout the drawings.

### DETAILED DESCRIPTION

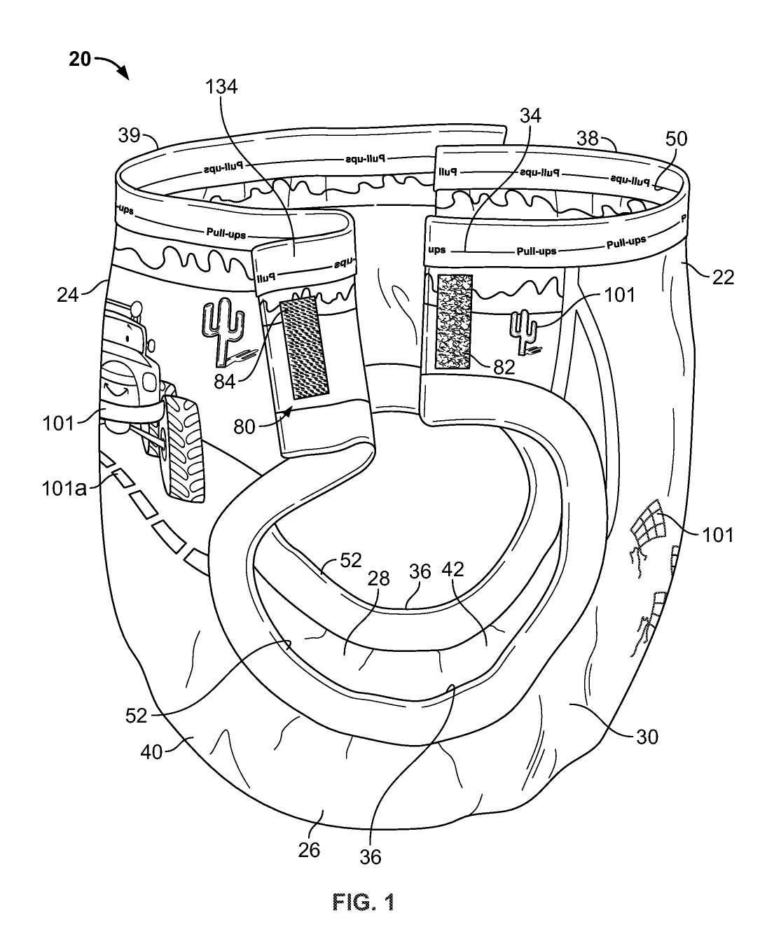

Referring now to the drawings and in particular to FIG. 1, an absorbent article according to one embodiment is representatively illustrated therein in the form of children's toilet training pants and is indicated in its entirety by the reference numeral 20. The absorbent article 20 may or may not be disposable, which refers to articles that are intended to be discarded after a limited period of use instead of being laundered or otherwise conditioned for reuse. It is understood that the present invention is suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of the various aspects of the embodiments described herein are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference.

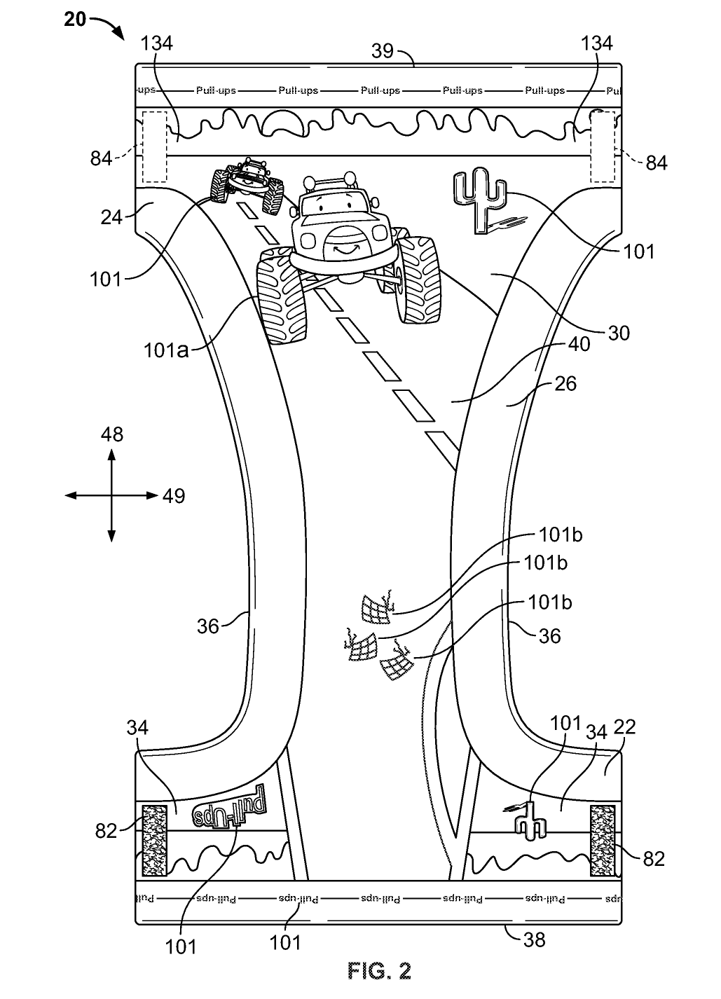

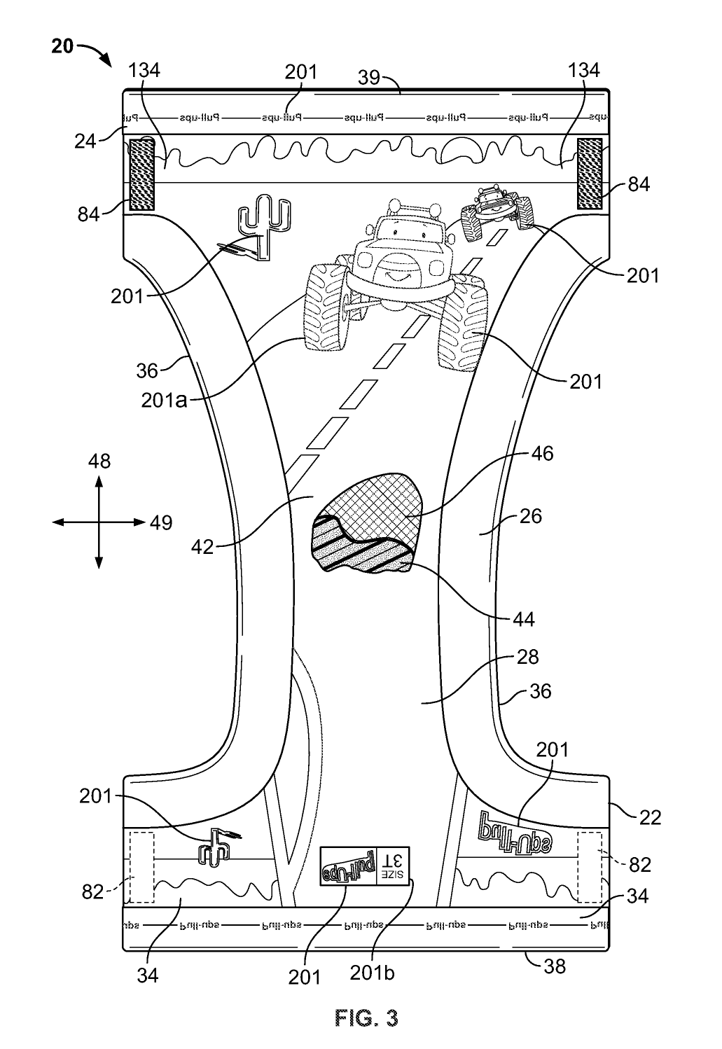

The pair of training pants 20 is illustrated in FIG. 1 in a partially fastened condition. The pants 20 define a longitudinal direction 48 and a lateral direction 49 perpendicular to the longitudinal direction as shown in FIGS. 2 and 3. The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The front and back waist regions 22, 24 include those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The pants 20 also define an inner surface 28 adapted in use (e.g., positioned relative to the other components of the pants 20) to face and be disposed toward the wearer, and an outer surface 30 opposed with the inner surface for facing away from the wearer.

As used herein, the terms inner and outer are used in reference to a direction taken perpendicular to the longitudinal and lateral directions of the pants 20 (i.e., the absorbent article), with the term inner being used to indicate a direction nearer to a wearer of the article and the term outer indicating a direction away from the wearer.

With additional reference to FIGS. 2 and 3, the pair of training pants 20 has a pair of laterally opposite side edges 36 and a pair of longitudinally opposite waist edges (broadly, longitudinal ends), respectively designated front waist edge 38 and back waist edge 39. Front and back side panels 34 and 134 of the pants, upon wearing thereof, include the portions of the training pants 20 which are positioned on the hips of the wearer. These front and back side panels 34 and 134 can be permanently bonded together to form the three-dimensional configuration of the pants 20, or be releasably connected with one another such as by the fastening system 80 as illustrated in FIG. 1. The side panels 34, 134 may be formed integrally with the rest of the training pants, or be formed separate therefrom and secured thereto by suitable bonding techniques, such as adhesive bonding, pressure bonding, thermal bonding and/or ultrasonic bonding.

The illustrated training pants 20 have an outer cover 40 (broadly, an outer layer or substrate of the pants) having an outer surface at least in part, and illustrated embodiment entirely defining the outer surface 30 of the training pants. A bodyside liner 42 (FIGS. 1 and 3) (broadly, an inner layer or substrate of the pants), having an inner surface at least in part and in the illustrated embodiment entirely defining the inner surface 28 of the pants, is in overlaid or opposed relationship with the outer cover. The liner 42 can be suitably joined to the outer cover 40 along at least a portion of the longitudinal ends 38, 39 of the pants 20 and/or along the side edges 36 thereof by any suitable means such as, without limitation, adhesive bonding, ultrasonic bonding, thermal bonding, and the like. An absorbent structure 44 (FIG. 3) is disposed between the outer cover 40 and the bodyside liner 42 for absorbing liquid body exudates exuded by the wearer.

With the training pants 20 in a fastened position as partially illustrated in FIG. 1, the front and back waist regions 22, 24 are connected together by the fastening system 80 to define the three-dimensional pants configuration having a waist opening 50 and a pair of leg openings 52. The front and back waist edges 38 and 39 (e.g., longitudinal ends) of the training pants 20 are configured to encircle the waist of the wearer to define the waist opening 50 (FIG. 1) of the pants.

The illustrated fastening system 80 includes laterally opposite first fastening components 82 adapted for refastenable engagement to corresponding laterally opposite second fastening components 84. In one aspect, a surface of each of the fastening components 82, 84 includes a plurality of engaging elements. The engaging elements of the first fastening components 82 are adapted to repeatedly engage and disengage corresponding engaging elements of the second fastening components 84 to releasably secure the pants 20 in its three-dimensional configuration. The fastening components 82, 84 can include any refastenable fasteners suitable for absorbent articles, such as adhesive fasteners, cohesive fasteners, mechanical fasteners, or the like. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

The outer cover 40 suitably comprises a material layer that is substantially liquid impermeable. For example, the outer cover 40 may comprise a single layer of liquid impermeable material, or more suitably a multi-layered laminate structure in which at least one of the layers is liquid impermeable. For instance, the outer cover 40 can comprise a liquid permeable outer layer and a liquid impermeable inner layer that are suitably joined together by a laminate adhesive, ultrasonic bonds, thermal bonds, pressure bonds or the like. The outer layer may also be made of those materials of which the liquid permeable bodyside liner 42 is made. While it is not a necessity for the outer layer to be liquid permeable, it is suitable that it provides a relatively cloth-like texture to the wearer.

The inner layer of the outer cover 40 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outer cover 40 when a single layer, prevents waste material from wetting articles, such as bed sheets and clothing, as well as the wearer and caregiver.

Alternatively, the outer cover 40 may include a woven or non-woven fibrous web layer that has been totally or partially constructed or treated to impart the desired levels of liquid impermeability to selected regions that are adjacent or proximate the absorbent structure. The outer cover 40 may also be stretchable, and in some aspects it may be elastomeric. Reference is made to U.S. Pat. No. 5,883,028, issued to Morman et al., U.S. Pat. No. 5,116,662 issued to Morman and U.S. Pat. No. 5,114,781 issued to Morman, all of which are hereby incorporated herein by reference, for additional examples of suitable outer cover 20 materials.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent structure 44. As an example, one suitable liquid permeable bodyside liner 42 is a nonwoven polyethylene/polypropylene bicomponent web having a basis weight of about 27 gsm; the web may be spunbonded or a bonded carded web. Alternatively, the bodyside liner 42 may be stretchable, and in some aspects it may be elastomeric. Reference is made to U.S. patent application Ser. No. 09/563,417 filed on May 3, 2000 by Roessler et al., U.S. patent application Ser. No. 09/698,512 filed on Oct. 27, 2000 by Vukos et al., both of which are incorporated by reference herein, for additional information regarding suitable bodyside liner materials.

The absorbent structure 44 is disposed between the outer cover 40 and the bodyside liner 42. The absorbent structure 44 in one suitable embodiment extends longitudinally from the crotch region 26 into both the front and back waist regions 22 and 24. It is contemplated, however, that the absorbent structure 44 may extend from the crotch region 26 into only the front waist region 22, or only the back waist region 24, without departing from the scope of this invention.

The absorbent structure 44 can be suitably compressible, conformable, non-irritating to a wearer's skin, and capable of absorbing and retaining liquids and certain body wastes. For example, the absorbent structure 44 may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof.

The materials may be formed into an absorbent web structure by employing various conventional methods and techniques known in the art. For example, the absorbent structure 44 may be formed by a dry-forming technique, an air forming technique, a wet-forming technique, a foam-forming technique, or the like, as well as combinations thereof. Methods and apparatus for carrying out such techniques are well known in the art. The absorbent structure 44 may alternatively include a coform material such as the material disclosed in U.S. Pat. Nos. 4,100,324 to Anderson, et al.; 5,284,703 to Everhart, et al.; and 5,350,624 to Georger, et al.; which are incorporated herein by reference.

The term “coform material” generally refers to composite materials comprising a mixture or stabilized matrix of thermoplastic fibers and a second non-thermoplastic material. As an example, coform materials are made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene.

Superabsorbent material may be suitably present in the absorbent structure 44 in an amount of from about 0 to about 90 weight percent based on total weight of the absorbent structure. The absorbent structure 44 may suitably have a density within the range of about 0.10 to about 0.35 grams per cubic centimeter. Superabsorbent materials are well known in the art and can be selected from natural, synthetic, and modified natural polymers and materials.

In one aspect, the absorbent structure 44 may be stretchable so as not to inhibit the stretchability of other components to which the absorbent structure may be adhered, such as the outer cover 40 and bodyside liner 42. For example, the absorbent structure may include materials disclosed in U.S. Pat. Nos. 5,964,743, 5,645,542, 6,231,557, 6,362,389, and international patent application WO 03/051254, the disclosure of each of which is incorporated by reference herein. Optionally, a substantially liquid permeable wrapsheet (not shown) may surround the absorbent structure 44 to help maintain the integrity of the absorbent structure 44.

A surge management layer 46 (FIG. 3) may be disposed between the absorbent structure 44 and the liner 42, and may or may not be attached to various components of the article 20 such as the absorbent structure and/or the bodyside liner 42. A surge management layer 46 helps to decelerate and diffuse surges or gushes of liquid that may be rapidly introduced into the absorbent structure 44 of the article 20. Desirably, the surge management layer 46 can rapidly accept and temporarily hold the liquid prior to releasing the liquid into the storage or retention portions of the absorbent structure 44. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are incorporated by reference herein. It is understood, however, that the surge management layer 46 may be omitted and remain within the scope of this invention.

As shown in FIGS. 1 and 2, the training pants 20, and in particular the outer cover 40 thereof, has one or more graphic elements 101 (referred to particularly herein as outer graphic elements) applied thereto. Examples of such outer graphic elements 101 include, but are not limited to, scenes, characters, animals, objects, alphanumerics such as numbers, letters, words, phrases and the like, highlighting or emphasizing of leg and waist openings 52, 50 in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product. The outer graphic elements 101 are suitably positioned on the training pants 20 at predetermined locations, e.g. relative to the longitudinal and lateral directions of the pants 20.

The outer graphic elements 101 may be applied to the pants 20 using a suitable printing process, such as a flexographic printing process according. Flexographic printing apparatus and processes are known to those skilled in art and need not be further described herein. For example such apparatus are shown and/or described in U.S. Pat. Nos. 5,458,590 (Schleinz et al.); 5,566,616 (Schleinz et al.); U.S. 2003/0019374A1 (Harte); and 4,896,600 (Rogge et al.). Alternatively, the outer graphic elements 101 may be printed, sprayed, or otherwise applied to the training pants 20 by another suitable printing method (e.g., ink jet, rotogravure, etc.) or by combinations thereof, such as by applying the graphic elements 101 to the pants 20 in different printing stages.

In the illustrated embodiment of FIGS. 1 and 2, one or more of the outer graphic elements 101 is suitably disposed on the training pants 20 so as to be visible from exterior of the pants—thereby resembling a conventional cloth pair of underwear or other garment. The term “visible from exterior of the pants” as used herein means visible while looking at the outer surface 30 of the pants 20 in a direction from the outer surface to the inner surface 28 of the pants. The term “visible” is used here to mean visible to an unaided human eye with 20-20 (natural or corrected) vision from a distance typically incurred during normal use of the pants, such as by the wearer during donning of the pants. In one particularly suitable embodiment one or more of the outer graphic elements 101 are applied to the outer cover 40, and more suitably to the outer surface of the outer cover (and hence to the outer surface 30 of the training pants 20). It is understood, however, that one or more of the outer graphic elements 101 may be applied other than to the outer facing surface of the outer cover 40, such as to the inner facing surface of the outer cover, or where the outer cover is of multiple layers of material one or more of the outer graphic elements 101 may be applied to the inner and/or outer surface of any one or more of the layers of such an outer cover without departing from the scope of this invention. It is also contemplated that one or more of the outer graphic elements 101 may be applied to a training pants 20 component other than the outer cover 40, such as to an outer facing surface of the absorbent structure 44, as long as the outer graphic element is visible from exterior of the training pants 20.

With particular reference now to FIG. 3, the training pants 20 also comprises one or more inner graphic elements 201 applied to the training pants so as to be separate from the one or more outer graphic elements 101 and visible from interior of the pants 20. The term “visible from interior of the pants” as used herein means visible while looking at the inner surface 28 of the pants 20 in a direction from the inner surface to the outer surface 30 of the pants. Examples of such inner graphic elements 201 include, but are not limited to, scenes, characters, animals, objects, alphanumerics such as numbers, letters, words, phrases and the like, highlighting or emphasizing of leg and waist openings 52, 50 in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product. The inner graphic elements 201 are suitably disposed on the training pants 20 at predetermined locations, e.g., relative to the longitudinal and lateral directions of the pants.

In the illustrated embodiment of FIG. 3, at least one of the inner graphic elements 201 is substantially identical to a corresponding one of the outer graphic elements 101 and more suitably is also superposed with the corresponding one of the outer graphic elements to further enhance the resemblance of the pants to a conventional cloth pair of underwear or other garment. For example, one of the outer graphic elements 101 in FIG. 2 comprises an outer car design, designated 101a. A corresponding one of the inner graphic elements 201 in FIG. 3 comprises an inner car design 201a that is separate from and substantially identical to the outer car design. More suitably, the outer car design 101a and the inner car design 201a are located on the training pants 20 (relative to the longitudinal and lateral directions 48, 49 of the pants) in substantially superposed relationship with each other so as to convey the image that the car design is a single image present through the thickness of the pants, e.g., in the manner of conventional underwear or other undergarments.

In the illustrated embodiment of FIGS. 2 and 3, the inner car design 201a appears as a reverse, or mirror image of the outer car design 101a. It will be understood that this is for illustrative purposes only, i.e., with one looking at the outer car design 101a in FIG. 2 from exterior of the pants 20 and looking at the inner car design 201a in FIG. 3 from the opposite direction—from interior of the pants. As viewed from a common direction through the thickness of the fully assembled pants 20 (assuming one could see through the pants), however, the inner and outer car designs 201a, 101a would generally overlay each other.

The term “superposed” as used in reference to the inner and outer graphic elements 201, 101 is intended to refer to the relative longitudinal and lateral locations of an inner graphic element and a corresponding, substantially identical outer graphic element irrespective of whether the graphic elements are in direct overlaid relationship with each other (such as where the liner 42 and the outer cover 40 are adhered to each other, e.g., adjacent the front and/or back waist edges 38, 39 of the illustrated pants 20), or spaced from each other by a gap therebetween, or separated from each other by other components disposed between (in the thickness direction) the inner graphic element and corresponding outer graphic element. For example, in the illustrated embodiment of FIGS. 1-3 the absorbent structure 44 disposed between the liner 42 and outer cover 40 of the pants 20 may be disposed between the inner car design 201a and the outer car design 101a and yet the inner and outer car designs are considered herein to be in superposed relationship with each other.

It is also understood that the inner graphic element 201 and corresponding, substantially identical outer graphic element 101 need not be in precise superposed relationship with each other to remain within the scope of this invention. For example, due to printing, manufacturing and substrate alignment tolerances, and/or due to stretching, bunching, wrinkling, flexing or other relative movement of one or more components of the pants, the inner graphic element 201 and corresponding outer graphic element 101 may not be at the exact same longitudinal and lateral locations of the pants. The term superposed is intended to mean that the inner graphic element 201 and corresponding outer graphic element 101 are sufficiently located relative to each other to convey the general appearance of a single graphic that extends through the thickness of the pants.

The term “substantially identical” as used in reference to the inner graphic element 201 being substantially identical to the corresponding outer graphic element 101 is intended to mean that the inner and outer graphic elements are substantially identical in at least shape, size and image irrespective of color and/or line weight. That is, the inner graphic element 201 may appear different from the outer graphic 101 in at least one characteristic relating to color or line weight and still be considered substantially identical to the corresponding outer graphic element within the scope of this invention.

In one particularly suitable embodiment one or more of the inner graphic elements 201 is applied to the bodyside liner 42 (broadly, the inner layer or substrate) of the pants 20, and more suitably to the outer surface of the bodyside liner. It is understood, however, that one or more of the inner graphic elements 201 may be applied other than to the outer surface of the liner 42, such as to the inner surface of the liner, or to a training pants component other than the liner such as to an inner facing surface of the surge management layer 46, the absorbent structure 44 and/or the tissue wrap surrounding the absorbent structure as long as the inner graphic element is visible from interior of the training pants 20.

As seen best in FIGS. 1 and 2, a plurality of the inner graphic elements 201 are substantially identical to and superposed with a corresponding plurality of the outer graphic elements 101 to provide a themed or patterned absorbent article (i.e., training pants 20) to further provide a more conventional underwear or garment appearance. Such an arrangement also better masks the visibility of the absorbent structure 44 and other internal components of the pants 20 to thereby facilitate the desired appearance of conventional underwear or other garment.

In the illustrated embodiment of FIG. 2, at least one of the inner graphic elements 201, and more suitably each of the inner graphic elements that corresponds to a respective one of the outer graphic elements 101, appears faded relative to (e.g., as compared to) its corresponding outer graphic. For example, referring again to the inner and outer car designs 201a, 101a of FIGS. 2 and 3, the inner car design appears faded relative to the appearance of the outer car design. Such a faded appearance of the inner graphic element 201 conveys the image of a single graphic applied to the outer surface of a conventional undergarment and seen from interior of the undergarment through the thickness thereof so as to provide a more undergarment-like appearance to the pants. The term “faded” as used in reference to the one or more inner graphic elements 201 means that the inner graphic element as viewed from interior of the training pants appears less bright or visible than the outer graphic element 101 as viewed exterior of the pants.

In one embodiment, the relative faded appearance of the at least one inner graphic element 201 is achieved by varying at least one characteristic of the color(s) used in applying the inner graphic element to the pants 20 as compared to the same characteristic used in applying the outer graphic element 101 to the pants. For example, in one suitable embodiment the inner graphic element 201 may have a color tint that is different from a color tint of the corresponding, substantially identical outer graphic element 101, and more suitably a lower tint. More suitably, the inner graphic element 201 may have a tint that is in the range of about 5 percent to about 80 percent of the tint of the corresponding outer graphic element 101, more suitably about 20 percent to about 50 percent, and even more suitably about 40 percent of the tint of the corresponding outer graphic element.

In another suitable embodiment, the inner graphic element 201 may have a different, and more suitably lower, ink coverage area than that of the corresponding outer graphic element 101. The ink coverage area refers to that percentage of a given planar area of the pants 20 (e.g., in the illustrated embodiment, the liner 42 for the inner graphic element and the outer cover 40 for the outer graphic element) that is covered by ink of the respective inner and outer graphic element. For example, where the inner and outer graphic elements 201, 101 are applied by flexographic dot printing and/or flexographic process printing, the inner graphic element may have a lower dot density (i.e., number of dots per unit area) or other ink density than the corresponding outer graphic element and/or the inner graphic element may be applied using smaller dots or less ink than those used in applying the corresponding outer graphic element. In one particularly suitable embodiment, the ink coverage area of the inner graphic element 201 is suitably in the range of about 5 percent to about 50 percent of the ink coverage area of the corresponding outer graphic element 101, more suitably about 10 percent to about 40 percent, still more suitably about 10 percent to about 30 percent, and even more suitably about 20 percent of the ink coverage area of the corresponding outer graphic element.

In another suitable embodiment, the inner graphic element 201 has different color density than that of the substantially identical, corresponding outer graphic element 101, and more suitably a lower color density. The color density of a graphic element, as used herein, refers to the density of the color produced by the ink on the substrate to which the ink is applied. The human eye assessment of the strength of a color is not related to the reflectance in a linear manner, but is rather more nearly a logarithmic relationship. More specifically, color density is empirically defined by the equation:

Density=log₁₀(I/R)

where;

I is the intensity of incident light and

R is the intensity of reflected light.

A lower color density equates to a lower vibrancy or intensity of the color. In particular, as used herein, the color density of the graphic element refers to the color density of the dominant primary color of a graphic element (e.g., yellow, magenta, cyan or black) as determined using a densitometer in accordance with the Color Density Test set forth below. Suitably, the color density of the dominant primary color of the inner graphic element 201 is less than the color density of the dominant primary color of the substantially identical, corresponding outer graphic element 101. More suitably, the color density of the inner graphic element 201 is in the range of about 20 percent to about 60 percent of the color density of the corresponding outer graphic element 101, and even more suitably about 40 percent of the color density thereof.

### Color Density Test

The following test is used to determine the color density of the dominant primary color in a graphic element applied to a substrate. The test involves using a reflectance densitometer, and more particularly a densitometer available from X-Rite, Inc. of Grandville, Mich., U.S.A. as model designation X-RITE 508 Densitometer. The densitometer is set for Status T measurement (which filters the density data in accordance with ANSI Status T Computerized Color Response) and for measuring the color density of the dominant primary color of a sample.

A sample substrate having a graphic element applied thereto should be at least 6 mm in diameter (or in both length and width where the sample is non circular). The sample is placed on a white measuring tile and the densitometer is placed over the sample, with the cross-hairs of the densitometer sole plate positioned over the graphic element. The nose of the densitometer is then held down against the sample until the display reads “Completed.” The measured color density is displayed by the densitometer as a numeric value (dimensionless) preceded by a letter corresponding to the dominant primary color of the measured graphic element; the letter Y referring to yellow, the letter M referring to magenta, the letter C referring to cyan, and the letter V referring to black.

At least three measurements should be taken and the results averaged to determine the color density of the sample being measured.

In other embodiments, the relative faded appearance of the inner graphic element 201 is achieved by applying the inner graphic element other than to the inner surface of the bodyside liner (i.e., other than to the inner surface 28 of the pants 20) so that the inner graphic element as viewed from interior of the pants is seen through the liner. For example, the inner graphic element 201 may be applied to the outer facing surface (the surface that faces toward the outer cover 40) of the liner 42, or it may be applied to the surge management layer 46, to the absorbent structure 44 or other component of the pants 20 as long as the inner graphic element is separate from the corresponding outer graphic element and is visible as viewed interior of the pants (i.e., the absorbent article).

Each of the inner and outer graphic elements 201, 101 in accordance with one embodiment are suitably permanent graphics, i.e., applied using a permanent ink. The term “permanent graphic” is used herein to refer to a graphic element that does not substantially change its degree of visibility when the absorbent article is insulted with urine in simulated use conditions. The change in visibility of a graphic element or a portion of a graphic element can be determined based on a person's observation of the graphic element before and after the article containing the graphic element is exposed to liquid.

For purposes hereof, an article is exposed to liquid by immersing the article completely in an aqueous solution containing 0.9 weight percent sodium chloride, used at room temperature (≅23° C.), for a period of twenty minutes. After 20 minutes the product is removed from the aqueous solution and placed on a TEFLON™ coated fiberglass screen having 0.25 inch (6.35 mm) openings, which is commercially available from Taconic Plastics Inc., Petersberg, N.Y., USA, which in turn is placed on a vacuum box and covered with a flexible rubber dam material. A vacuum of 3.5 kilopascals (0.5 pounds per square inch) is drawn in the vacuum box for a period of 5 minutes, after which the article is removed and observed. A person with normal or corrected vision of 20-20 should make the observations from a distance of 1 meter in an environment providing 30 footcandles (320 Lux) of illumination. Changes in the visibility of the graphic element should be identified, and distinguished where necessary from changes in the color of other materials such as fluff pulp within an absorbent structure.

It is contemplated, however, that one or more of the inner graphic elements 201 and/or one or more of the outer graphic elements 101 may comprise an active graphic for providing a caregiver and/or the wearer of the absorbent article 20 a signal that a liquid insult has occurred. As used herein, the term “active graphic” refers to a graphic element that visibly (to an unaided human eye having 20-20 vision) changes appearance in response to a liquid insult of the article. For example, such an active graphic may fade or otherwise appear faint following liquid insult (often referred to as a fading graphic), or the active graphic may change from a relatively faint appearance to a bolder, or at least more readily visible appearance (often referred to as an appearing graphic), or the active graphic may change colors in response to a liquid insult. In the illustrated embodiment of FIG. 2, for example, outer graphic elements 101 in the form of flags 101b are suitably active graphics, and more suitably fading graphics. Examples of active graphics suitable for use with the training pants 20 or other absorbent articles within the scope of this invention are described in U.S. Pat. No. 6,297,424 issued Oct. 2, 2001 to Olson, et al. and U.S. Pat. No. 6,307,119 issued Oct. 23, 2001 to Cammarota et al., the entire disclosures of which are incorporated by reference herein.

It is also understood that one or more of the inner graphic elements 201 need not correspond with a substantially identical, superposed outer graphic element 101. In such an embodiment, the inner graphic elements 201 suitably comprise at least one first inner graphic element that corresponds to a substantially identical superposed outer graphic element 101 and at least one second inner graphic element separate from the at least one first inner graphic element with the pants 20 (i.e., the absorbent article) being substantially free from an outer graphic element that is substantially identical to and in superposed relationship with the second inner graphic element. For example, in the illustrated embodiment of FIGS. 2 and 3, while the inner car design 201a is superposed with the substantially identical outer car design 101a, a second inner graphic element that has the appearance of a garment label 201b (e.g., a label that indicates size and/or other garment information) is applied to the pants 20 but no such corresponding outer graphic element is present.

Likewise, one or more of the outer graphic elements 101 may not correspond with a substantially identical, superposed inner graphic element 201. In such an embodiment, the outer graphic elements 101 suitably comprise at least one first outer graphic element that corresponds to a substantially identical superposed inner graphic element 201 and at least one second outer graphic element separate from the at least one first outer graphic element with the pants 20 (i.e., the absorbent article) being substantially free from an inner graphic element that is substantially identical to and in superposed relationship with the second outer graphic element. For example, in the illustrated embodiment of FIGS. 2 and 3 the flags 101b (i.e., the active graphics) visible as outer graphic elements 101 in FIG. 2 are omitted from the pants 20 in FIG. 3.

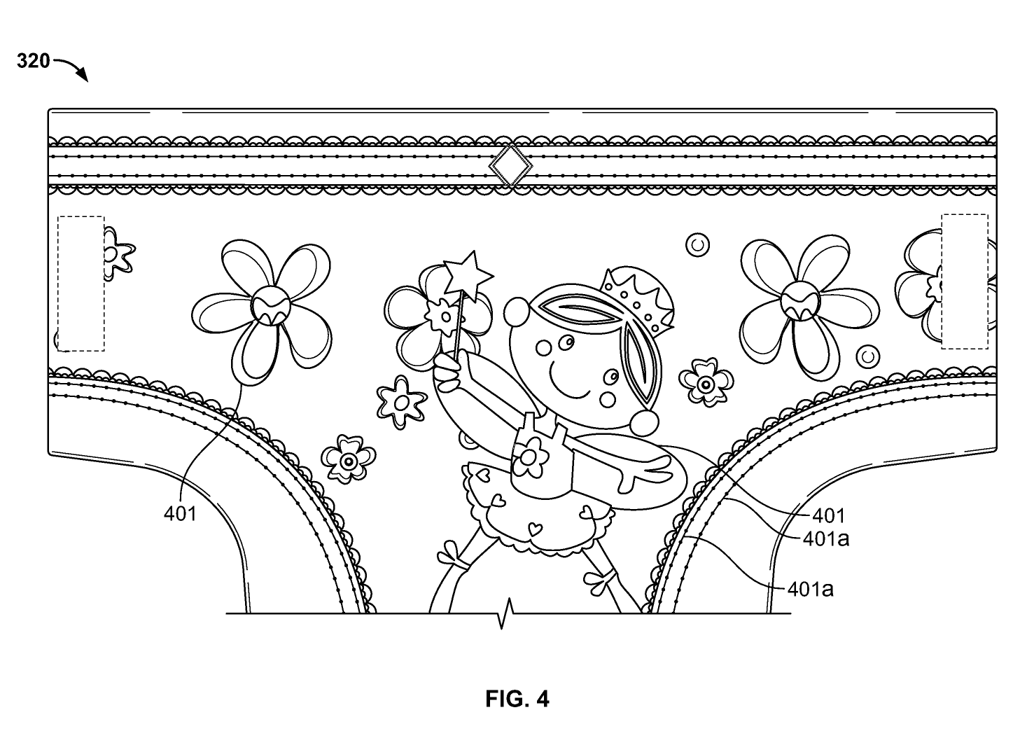

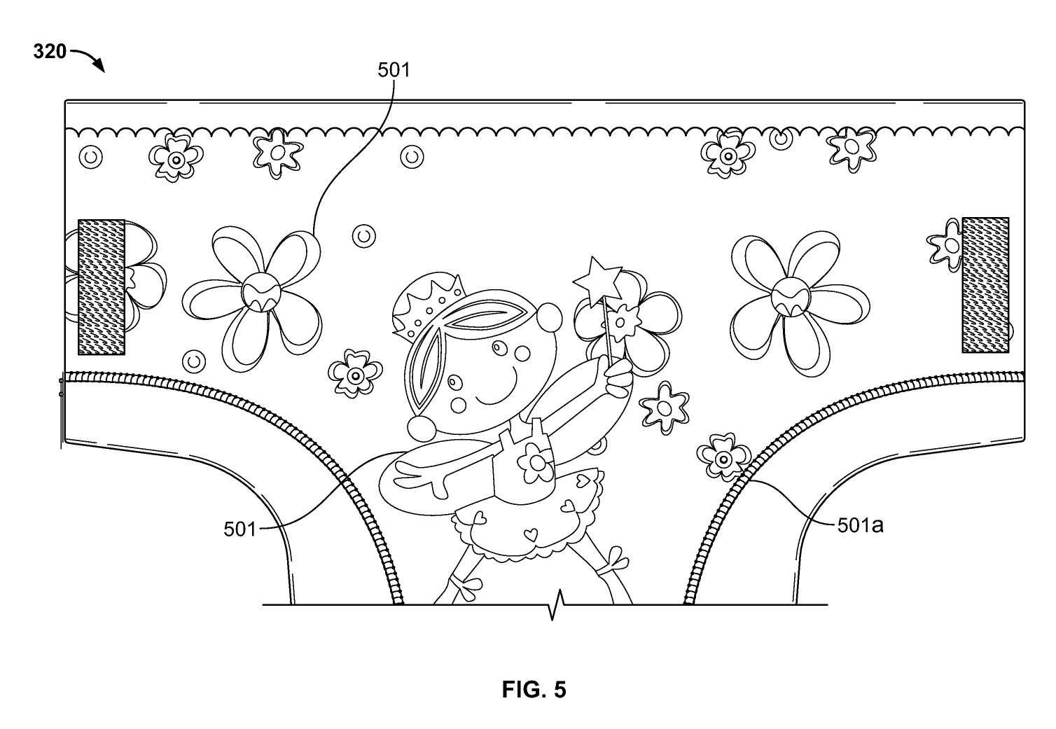

In accordance with one method of making an absorbent article having inner and outer graphic elements thereon, such as the pants 20 of FIGS. 1-3 (and those of FIGS. 4-5 as well), the outer graphic elements (first graphic elements) are applied to a first substrate (such as the outer cover 40 in the illustrated embodiment) and the inner graphic elements (second graphic elements) are applied to a second substrate (such as the liner 42 in the illustrated embodiment). The inner graphic elements may be applied to the second substrate using a printing process similar to that used to apply the outer graphic elements to the first substrate, or by a suitable other printing process as described previously. The first and second substrates are then assembled in opposed relationship with each other, with the absorbent structure 44 disposed therebetween, and secured in the assembled configuration, such as the training pants 20 of FIGS. 1-3. In assembling the first and second substrates in opposed relationship, the first (inner) graphic elements are superposed (or at least associated as discussed below in connection with the embodiment of FIGS. 4 and 5) with the second (outer) graphic elements.

FIGS. 4 and 5 illustrate another embodiment of training pants 320 similar to that of FIGS. 1-3, with one or more inner graphic elements 501 being substantially identical to and superposed with a corresponding outer graphic element 401. Additionally, in this embodiment, at least one outer graphic element 401 is different from but otherwise associated with a corresponding inner graphic element 501 such that the outer and inner graphic element together provide the appearance of a single feature of conventional underwear or other garment that extends through the thickness thereof so as to be visible from the interior and exterior of the pants 320. The term “associated” as used in reference to the inner and outer graphic elements 501, 401 of this embodiment means that the inner graphic element is generally at or near the same longitudinal and lateral position as (but not necessarily superposed with) the outer graphic element and is related to the outer graphic element in some common thematic or pattern characteristic.

For example, as seen in FIG. 4 (which is a view from exterior of the pants) one of the outer graphic elements 401 appears in the form of an outer stitching pattern 401a that runs adjacent the side edges of the pants. In this particular embodiment, the outer stitching pattern 401a is formed using a combination of pattern or point bonding with graphic elements extending between the bond points to give the appearance of threaded stitching. It is understood, however that the outer stitching pattern 401a may be formed entirely as a graphic element and remain within the scope of this embodiment.

Interior of the pants 320, as seen in FIG. 5, one of the inner graphic elements 501 appears in the form of an inner stitching pattern 501a that is associated with the outer stitching pattern 401a. That is, the inner stitching pattern 501a resembles or is intended to convey the appearance of what a conventional stitching pattern would look like if actual stitching was applied through the thickness of the pants 320.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. An absorbent article for personal wear, the absorbent article comprising;
an inner surface for facing a wearer of said absorbent article;
an outer surface in generally opposed relationship with the inner surface for facing away from said wearer;
an absorbent structure disposed between the inner surface and the outer surface of the absorbent article;
an outer graphic element visible from exterior of the absorbent article; and
an inner graphic element visible from interior of the absorbent article, the inner graphic element being separate from the outer graphic element and substantially identical thereto, said inner and outer graphic elements being in superposed relationship with each other on the absorbent article.

2. The absorbent article set forth in claim 1 wherein the inner graphic element has a generally faded appearance relative to the outer graphic element.

3. The absorbent article set forth in claim 1 wherein the absorbent article comprises an outer cover at least in part defining the outer surface of the absorbent article, the outer graphic element being disposed on the outer cover of the absorbent article.

4. The absorbent article set forth in claim 1 wherein the absorbent article comprises a liner having an inner surface at least in part defining the inner surface of the absorbent article, and an outer surface facing the outer surface of the absorbent article, the inner graphic element being disposed on the liner.

5. The absorbent article set forth in claim 1 wherein the inner graphic element is disposed on the outer surface of the liner.

6. The absorbent article set forth in claim 2 wherein the outer graphic element has a tint, the inner graphic element having a tint that is substantially less than the tint of the outer graphic element.

7. The absorbent article set forth in claim 6 wherein the tint of the inner graphic element is about 5 percent to about 80 percent of the tint of the outer graphic element.

8. The absorbent article set forth in claim 2 wherein the outer graphic element has an ink coverage area, the inner graphic element having an ink coverage area that is substantially less than that of the outer graphic element.

9. The absorbent article set forth in claim 6 wherein the inner graphic element has an ink coverage area of about 5 to about 50 percent of the ink coverage area of the outer graphic element.

10. The absorbent article set forth in claim 2 wherein the outer graphic element has an ink color density, the inner graphic element having an ink color density that is substantially less than that of the outer graphic element.

11. The absorbent article set forth in claim 1 wherein the inner graphic element is a first inner graphic element, the outer graphic element being substantially identical to and in superposed relationship with the first inner graphic element, the absorbent article further comprising a second inner graphic element separate from the first inner graphic element and visible from interior of the absorbent article, the absorbent article being free from an outer graphic element that is substantially identical to and in superposed relationship with the second inner graphic element.

12. The absorbent article set forth in claim 11 wherein the second inner graphic element has the appearance of a garment label.

13. The absorbent article set forth in claim 1 wherein the outer graphic element is a first outer graphic element, the inner graphic element being substantially identical to and in superposed relationship with the first outer graphic element, the absorbent article further comprising a second outer graphic element separate from the first outer graphic element and visible from exterior of the absorbent article, the absorbent article being free from an inner graphic element that is substantially identical to and in superposed relationship with the second outer graphic element.

14. The absorbent article set forth in claim 13 wherein the second outer graphic element comprises an active graphic.

15. The absorbent article set forth in claim 1 wherein the absorbent article comprises training pants having an outer cover, the outer cover having an outer surface at least in part defining the outer surface of the training pants, and a liner in generally opposed relationship with the outer cover and having an inner surface at least in part defining the inner surface of the training pants, the absorbent structure being disposed between the liner and the outer cover, the inner graphic element being disposed on the training pants other than on the inner surface of the liner.

16. An absorbent article for personal wear, the absorbent article having a longitudinal direction and a lateral direction, said absorbent article comprising;
an inner layer having an inner surface for facing a wearer of the absorbent article;
an outer layer in opposed relationship with the inner layer and having an outer surface for facing away from said wearer;
an absorbent structure disposed between the inner layer and the outer layer;
an outer graphic element visible from exterior of the absorbent article; and
an inner graphic element separate from the outer graphic element and visible from interior of the article, the inner graphic element being associated with the outer graphic element, said inner and outer graphic elements being generally at the same longitudinal and lateral position on the absorbent article.

17. The absorbent article set forth in claim 16 wherein the inner and outer graphic elements each comprise a stitching pattern.

18. The absorbent article set forth in claim 16 wherein the inner graphic element has a generally faded appearance relative to the outer graphic element.

19. The absorbent article set forth in claim 16 wherein the absorbent article comprises an outer cover at least in part defining the outer surface of the absorbent article, the outer graphic element being disposed on the outer cover of the absorbent article.

20. The absorbent article set forth in claim 16 wherein the absorbent article comprises a liner having an inner surface at least in part defining the inner surface of the absorbent article, and an outer surface facing the outer surface of the absorbent article, the inner graphic element being disposed on the liner.

21. The absorbent article set forth in claim 16 wherein the inner graphic element is disposed on the outer surface of the liner.

22. An absorbent article for personal wear, the absorbent article comprising;
a liner having an inner surface for facing a wearer of the absorbent article;
an outer cover in opposed relationship with the liner and having an outer surface for facing away from said wearer;
an absorbent structure disposed between the inner layer and the outer layer;
an outer graphic element disposed on the outer cover; and
an inner graphic element separate from and substantially identical to the outer graphic element, the inner graphic element being disposed on the liner and being in generally superposed relationship with the outer graphic element.

23. The absorbent article set forth in claim 22 wherein the inner graphic element is disposed on the outer surface of the liner.

24. A method of making an absorbent article having an inner surface for facing a wearer of the absorbent article and an outer surface for facing away from the wearer, said method comprising:
applying a first graphic element to a first substrate of the absorbent article;
applying a second graphic element to a second substrate of the absorbent article, said second graphic element being substantially identical to the first graphic element; and
assembling the absorbent article with the first and second substrates in generally opposed relationship with each other and an absorbent structure disposed between the first and second substrates, the assembling of the first and second substrates being such that the first graphic element is in generally superposed relationship with the second graphic element.
